(19) United States
(12) Patent Application Publication
(10) Pub. No.: US 2009/0143672 A1
(43) Pub. Date: Jun. 4, 2009

(54) METHOD FOR MAPPING IMAGE REFERENCE POINTS TO FACILITATE BIOPSY USING MAGNETIC RESONANCE IMAGING

(76) Inventors: Steven E. Harms, Fayetteville, AR (US); Aiping Jiang, Lexington, MA (US); Xiaole Hong, Acton, MA (US)

Correspondence Address: DUANE MORRIS LLP - Philadelphia;IP DEPARTMENT, 30 SOUTH 17TH STREET, PHILADELPHIA, PA, 19103-4196, US

(21) Appl. No.: 12/328,644

(22) Filed: Dec. 4, 2008

Related U.S. Application Data
(60) Provisional application No. 60/992,218, filed on Dec. 4, 2007.

(51) Int. Cl. A61B 5/05

(52) U.S. Cl. 600/426; 382/131

(57) ABSTRACT

In NMR/MRI imaging, a location is noted for a point in the imaged space, and referred to a reference location so that the point in imaged space is known thereafter, without the need to locate the point again in further imaging steps. For breast cancer diagnosis and biopsy, a breast holding fixture immobilizes the breast. A volumetric image is taken encompassing a portion of the breast. In the same or a subsequent image, a fiducial mark is detected to determine the position of a holder for a biopsy tool or other modality. The tissue feature can be a tumor, cyst or tubal lesion, made temporarily visible in the image by perfusion with a contrast agent. After the contrast agent dissipates, the location of the tissue feature can still be determined by reference to the position of the fiducial marker, which is optional adjustable by post-imaging metered displacement.

### BACKGROUND OF THE INVENTION

1. Field of the Invention

The invention relates to magnetic resonance imaging (MRI), particularly for imaging and mapping tissue structures within the breast, and more particularly to facilitating accurate collection of biopsy tissue samples.

2. Related Art

Nuclear magnetic resonance imaging (NMR or MRI) is a well developed technique for providing images that can be encoded and displayed as image planes or slices through a volume of tissue, or as two dimensional views or projections of the volume. The data is collected by distinguishing the electromagnetic responses of a nuclei from different tissues, and their respective spatial positions in the imaged volume. The variations in local responses are mapped as corresponding variations in displayed luminance, color or other characteristics, applied to displays that are generated by the imaging system to represent an image of the internal tissue structures.

MRI is useful for visualizing tissue structures, and for revealing differences in tissue condition, such as fluid or blood concentration. Reasonably fast image collection techniques are known that can be used to note time changes from one image to another. In the context of the present disclosure, MRI can be used for visualizing tissue structures including potentially cancerous tumors and lesions, for diagnostic purposes, biopsy and the like. MRI has been used successfully to image many organs of the body. The technique is suitable for imaging the internal structures of the breasts in diagnosing breast cancer.

Typically, an MRI system first establishes a static magnetic field to bias the magnetic moments of nuclei in the imaged tissue to align to the field. Radio frequency (RF) signals that are tuned to selected element resonances are used as excitation and phase encoding signals. Magnetic fields applied as gradient fields select a spatial line or plane to be read out, and can be varied in a pattern to repetitively sweep the position of a local imaging line or plane. The excitation signals displace the nuclear magnetic moments of nuclei from their alignment with the static biasing field, for those nuclei that are selected because the nuclei resonate at the excitation frequency. Nuclei that are adjacent to one another spin in phase in response to the excitation, providing an echo signal that can be resolved by location. The nuclei go out of phase after a time period that is partly a characteristic of the selected element. The nuclei precess back into alignment with the biasing field over a time that also varies with the nuclear element and the chemical or physical environment in which the nuclei reside.

Useful images can be obtained by selecting for the concentration of an element, such as using the hydrogen resonant frequency to select for concentrations of water that may characterize potentially cancerous tumors or may identify the location of a lesion due to associated edema. It is also possible to select for an element in a negative sense, for example selectively to ignore concentrations of fat tissue that may camouflage tissue structures of interest. This can be accomplished by selecting for a characteristic resonance frequency of fat tissues and rendering the responsive areas dark in a slice display view, or transparent in the projection of a volume. Data collection on and off a given resonant frequency or set of resonant frequencies may be useful.

The MRI system has an RF transmitting coil for exciting the nuclei and an RF receiving coil to receive the electromagnetic signals of the nuclei at different positions where local magnetic moments are spinning synchronously and producing an echo signal responsive to their excitation. The receiving coil receives the signal that is processed through an amplifier and signal processor running Fast Fourier Transforms (FFT), by which amplitude and phase responses from local points in the imaged volume are spatially resolved as discrete values associated with points in the volume being imaged. The imaging system resolves the volume to a three dimensional matrix of data values that each represent the smallest resolution of which the system is capable (these points or values being known as “voxels”). The voxel data can be displayed in various ways that enable a physician or other practitioner to visualize the tissue structures or characteristics.

The frequency, phasing, gradient, intensity, and time variations of these aspects alter the way the image is produced and/or captured and can be used to generate various images along several planes wherein in one tissue structure or another may be highlighted by establishing conditions, selecting the excitation, weighting the response by data processing steps and similarly controlling the signals produced and the data collected.

Certain auxiliary devices and techniques have been developed to augment the imaging process, and the imaging process has been applied to assist in certain diagnostic techniques that go beyond imaging. Among other examples, contrast agents have been employed to perfuse tissues with paramagnetic compounds that have an affinity for certain targeted tissues such as breast ductal lesions and tumors. In a contrast series process, a contrast agent is injected and a sequence of magnetic resonance images are collected to note the affinity of the contrast agent for certain tissues. The contrast agent produces temporarily enhanced contrast of those tissues over surrounding tissues, which contrast fades as the contrast agent washes in and out the tissues generally.

As another example, breast holding devices have been developed that compress and/or immobilize the breast so that clear images can be captured, or successive images can be referenced readily to a common reference point, or when necessary, the breast can be held stationary during piercing with a biopsy collection cannula or the like to collect a tissue sample. One such device is disclosed in U.S. Pat. No. 7,171,256, which is incorporated herein by reference. The breast holding device of '256 patent comprises a base plate and opposed curved breast paddles that hold the breast in place. The paddles have grid-like openings that allow objects, such as a biopsy needle, to pass through to intersect with targeted tissue.

A challenge is presented with respect to guiding a biopsy needle to the desired point at which a tissue sample is to be collected. Inasmuch as the target tissue occupies a point in three dimensional space within the breast or other tissue, it is necessary to consider the relative position at which the biopsy needle is supported (e.g., a height adjustable gimbal or similar mounting), the angular trajectory along which the needle is to pierce the breast, and the displacement of the needle relative to its longitudinal axis, so as to pierce just to the necessary depth.

A known technique for assisting in determining locations and the like is to provide “fiducial markers.” At least one marker device, and preferably plural spaced devices, are located at predetermined positions relative to the MRI imaging apparatus, such as affixed to a patient support table. The devices each carry a position marking structure that is located within the volume to be encompassed by the image. The marking structure is composed of a material that appears distinctly in NMR/MRI images of the tissue, such as a ferrous material. The positions of the fiducial marker(s) reference the collected image to the known location of the position marking structure.

Fiducial markers thus define a spatial reference. A single marker defines at least one point and may be elongated (e.g., with orthogonal members). Two spaced point markers define a line, three point markers a plane, etc. The markers establish a coordinate system and a means to assess relative dimensions. Where a marker is at a known position relative to a biopsy needle support, the marker can be helpful in determining the trajectory and/or displacement of the biopsy needle that are needed to intersect a tissue structure that can be seen and located at a three dimensional point in a collected image where the marker is also visible.

Guidance for biopsy needles is a subject that is addressed to some extent in the prior art. Guided biopsy may involve NMR/MRI imaging to visualize the position of a tumor and to plan a trajectory of a biopsy tool to intersect the tumor. Follow-up imaging can be used to assess whether the tool was or is properly positioned.

For example, U.S. Pat. No. 6,904,305 discloses a guided biopsy procedure. The disclosed method requires the technician to image a breast that has been compressed in a breast holding restraint and to elect a tumor or lesion from the results of such imaging. The user deploys a needle-like biopsy tool held in a fixture. The tool is advanced by a longitudinal distance from a position where the tool is held on the fixture at an angle. The position of the fixture, the elevation and angle of the tool, and the distance of longitudinal advance are calculated to conform to a planned trajectory, after first ensuring that the tool is placed to be moved through an opening in the breast restraint, in the direction required. The tool is positioned in relation to the fiducial markers associated with the patient support structures including the breast holding restraint. The markers also appear in one or more corresponding NMR/MRI images of the breast, which theoretically enable the respective positions and range of motion of the tool to be coordinated with the position of the elected tumor or lesion.

The foregoing technique of fiducial marking and planning is theoretically effective but is unwieldy. The technique also can be unsure if the tissue targeted for biopsy is not readily visible in follow-up image sequences. This problem is likely to occur when attempting to biopsy a lesion that was only made to appear using a contrast agent to render the lesion visible in an image. After perfusion with a contrast agent, which may reveal a lesion or similar tissue structure of interest, washout of the contrast agent causes the contrast at the tissue structure to be lost and the detail used to identify the tissue structure is lost.

It might be possible to repeatedly perfuse the patient, but this is not recommended. Contrast agents as known in the art are chemical agents that often contain gadolinium (Gd) as a paramagnetic element. As are other lanthanides, gadolinium compounds can have low to moderate toxicity. There are contraindications with respect to patients on dialysis.

A contrast agent can be injected through IV. The contrast agent travels through vascular system, binds to various tissues and remains bound at different rates, such that for a limited time, tissue that has been bound by a contrast agent appears different in an MRI than tissue that has not been bound by the contrast agent. Accordingly, the image is enhanced. It would be advantageous if enhanced contrast could be extended and better exploited.

Apart from biopsy, it would be advantageous in general to provide a way to exploit contrast agents so that the enhancement of contrast that enables one to locate tissue structures can be extended, so that multiple tissue structures can be marked, and so that a marked point in a tissue structure could be noted and found again in successive images or from different viewing perspectives when desired, even after the contrast agent has faded away.

### SUMMARY OF THE INVENTION

In order to provide a more versatile and yet effective way to ensure that a biopsy needle holder (or a similar device for deploying a modality) can be accurately positioned relative to tissue that may be identified in an earlier image but currently not visible, the present disclosure proposes that the needle holder or a fiducial marker on the needle holder form a point of reference for positioning, aiming and deploying the modality so as to accurately intersect the target tissue. The needle holder need not be positioned relative to the patient table, or fixed in space, and instead can be moved. The needle holder is provided with a displacement measurement encoder, or alternatively is arranged such that its displacement in position from one image to another can be measured. Then provided that the breast remains in position, the change in position of the needle holder is accommodated. A new trajectory is calculated based on the new position of the needle holder, which is used as a movable reference, and the holder is aimed to deploy the modality to intersect the target tissue accurately.

It is an object of the present technology to determine, and to store addressing information referring to, at least one reference point in a three dimensional voxel data set defining an NMR/MRI image. The reference point in the data can be referenced to a point in the imaged volume, namely a point in a sampled portion of the tissues of a patient. The location of the reference point can be selected automatically or by manual data input in conjunction with imaging a movable reference marker (e.g., on a needle holder), or by a combination of automatic and manual processes.

Another object is to provide a technique whereby the same spatial point in an imaged volume can be found in two or more images obtained for that volume, even if the point is only visible in one of the images, and optionally even if the images are obtained from different points of view. A positioning frame of reference is defined in a manner that is applicable to the two or more images or alternatively it can be assured that the imaged subject is stationary relative to the imaging system. The position of any point in the imaged volume has a measurable displacement in three dimensions from any other point. Whereas the imaged subject remains stationary in the image, any point can be adopted as a positioning reference origin in an image. The holder for a biopsy needle or similar movable element can be used as a point of reference, provided that it is imaged and can be aimed according to its current location. Insofar as trajectory may be calculated from the holder to a targeted tissue location in one image, and thereafter the holder should be moved, the displacement from the previous location of the holder can be determined versus the previous position. Then a new trajectory can be calculated to intersect the same tissue location from a new starting point of the holder.

In one embodiment, a fiducial marker on the holder is caused to appear in both images. The fiducial marker defines a point in three dimensions that may be a different point relative to the imaging apparatus in the two images. Provided that the subject is stationary in the image, namely at the same location relative to the iso-center of the field gradient coils in an MRI system, the new location of the fiducial marker provides a new location point from which a trajectory is calculated to intersect the tissue target. It is not necessary that the tissue target be visible when determining the trajectory of the newly-positioned holder.

The foregoing arrangements use a movable fiducial marker associated with the needle holder, and a known relationship between the position of the target tissue in the first and second images. Preferably the target tissue is at the same position in the two images, but it is also possible to account for an offset if the amount and direction of the offset are known. In an embodiment wherein orientation and image size are changeable from one image to another, an additional marker can be provided and made visible to define not only a point but also an orientation and a distance scale to relate one image to another. For example, a detectable fiducial marker can have three orthogonally spaced features, or three orthogonally spaced fiducial marker points can be detectably fixed in position.

In addition to determining the position of the holder, which as noted can be movable, the imaging system can be deemed to define an origin and an orientation that remains fixed in a physical frame of reference in which the imaging system is located. The positioning reference point or origin, and also the orientation of the physical frame of reference in this embodiment, can be the gradient iso-center of the magnetic field coils. By establishing a positioning reference that is applicable to two or more images, a feature that may be visible temporarily at an arbitrary location in an imaged volume of tissue, can be found again, in a subsequent image, even under circumstances where the attribute that rendered the feature visible is no longer present.

A further object is to facilitate correlation of the location of a point in a patient's tissues, which point is identified by stored addressing information referring to a voxel element or group of elements in a first NMR/MRI image of the sampled patient tissue, with the location of the same point in the patient's tissues, as such tissues appear in second NMR/MRI image that at least partly encompasses the same tissues in which the point is located.

An advantageous application of the invention is to permit a target tissue location to be determined at a time when an image with sufficient contrast is available to identify one or more targeted tissue structures, and thereafter to remain in memory as a defined location. That point can then be found and displayed or otherwise employed in one or more other images of the same tissue sample. In the case of a movable needle holder for biopsy samples, a new and arbitrary location of the holder is determined after repositioning the holder, for example by noting the location of a fiducial marking on the holder in a new image. One or more fiducial marks can be mounted on a needle holder that is moved from one location to another for targeting the lesions. The trajectory (orientation and distance) from the point at which the needle is held to the defined and stored location of the tissue sample is calculated. The needle can be deployed on the newly computed trajectory and accurately intersects the target tissue. This technique is operable even if the targeted tissue is wholly invisible in the second image that shows the new position of the needle holder.

The invention is particularly apt for imaging structures within a mammalian and preferably-human breast, such as diagnosing and treating breast cancer using NMR/MRI pulse sequence and data collection techniques that produce contrast involving differences in the location and density of tissue types including water and fat. Relatively high contrast images can be obtained, especially revealing targeted tissue structures including ducts, ductal lesions, cysts, tumors, vasculature, edema and ischemia and the like, by collecting one or more images at a peak or high contrast time in the process of perfusion with a contrast agent such as a paramagnetic element. After that time, contrast diminishes with the washout of the contrast agent and the targeted tissue may become relatively less visible or even invisible in subsequent image collection iterations, except possibly by repeating the perfusion operation.

However, according to an inventive aspect, one or more reference locations such as the precise location of a targeted tissue structure at one or more voxel positions is determined, and stored in memory. Alternatively, locations having a predetermined spatial relationship to the reference location can be stored. A visual identifier such as crosshairs or a volume marker or a line with graduation marking representing length, or a reticule or similar marker can be superimposed on a display of subsequently collected MRI images, controllably turned on and off and caused to identify a corresponding location in another image. Alternatively, the location can be remembered for its coordinates alone.

The invention facilitates tissue collection biopsy using a tool that can be guided on a trajectory to intersect a stored location address and/or to a related location determined in one or more images wherein the tool location is determined and can be related to the stored location. This can be accomplished even though the targeted tissue structure may not be visible in the image, although its location is known to be the stored location address. The trajectory is determined as a vector from the tool location as determined in current image data versus the tissue location in stored location coordinates.

Accurate biopsy as well as other operations referenced to a location in the patient tissue are facilitated. It is not necessary to provide a new high contrast image of the targeted tissue structure in order to guide the biopsy tool, or in order to set up the origin, angle and trajectory of a tool holding biopsy fixture that guides the path of the tool in piercing the tissues to the selected point at or related to the reference voxel position or positions that were stored in memory. Provided that the patient tissue bears the same positioning relative to the imaging apparatus, the stored location of the target can be found in any of the images and used as a target during projection and viewing, magnification, and when aiming or positioning a modality that is sensitive to location such as tissue collection by biopsy, application of an injection at a targeted point, or targeted nuclear, thermal or sonic radiation at a targeted point.

One embodiment of the invention is a method practiced by restraining a patient's breast relative to a base plate and one or more restraining paddles or in a comparable device for fixing the position of the breast and optionally compressing the breast tissue, imaging the breast, and locating at least one point of interest in the image. The point of interest can be a tumor, lesion, unknown structure or arbitrary point within the volume of the breast. Preferably the image is temporarily enhanced in contrast in one way or another, although enhancement of contrast is not a condition to obtain the benefits of the inventive method. The location of a target is noted.

The image data display is advantageously derived from one or more image voxel matrices stored in a processor memory and mapped to a image memory wherein data values correspond to levels in a range of luminance or colors of different brightness and saturation. The display can be configured to present the data as representing the level of response in a contrast enhanced image, or as representing the spatial concentration of a given response. The display can be configured to present the MRI data in various ways including, but not limited to, projections, magnified areas, rotations, threshold filters, combinations of images by addition, subtraction or Boolean function, overlays, etc.

At least one point of interest is identified. Optionally, plural points of interest can be identified. The point of interest can be a voxel position in a matrix that is registered to the imaging apparatus. The point of interest can be selected by identifying the point of interest at an X-Y position in an image data slice, thereby identifying a voxel position in the image and an X-Y-Z location in the tissue. Alternatively, an X-Y position of a feature can be identified in a 2D projection of the imaged volume, for example using a computer mouse or other pointer such as movable crosshairs. Identifying a point in a 2D projection defines a line in the 3D volume. By rotating the projection to a different perspective where the feature can be seen and again identifying the X-Y position of the feature in the rotated 2D projection, the intersection or nearest intersection of the two defined lines can be regarded as a voxel position in the image corresponding to the 3D location of the feature within the tissues.

The location of the feature is stored in computer memory as the address of an XYZ point in a Cartesian coordinate system or as longitudinal displacement, angles and radii in a polar or spherical coordinate system. At least one and optionally a plurality of points of interest are identified by noting and storing data representing the location of one or more points that may be noted. Advantageously, the points of interest can be the location in the image, in display memory and in a corresponding spatial volume, of a tissue structure such as a tumor or a lesion, cysts, duct branches, vascular features or other location(s) of interest.

In an advantageous application, one or more locations of interest is obtained and recorded in computer memory as a step during a procedure comprising perfusion with a contrast agent that causes a tissue structure to show clearly at high contrast. Subsequent washout of the contrast agent can cause the contrast associated with the tissue structure to fade away. However by that time the location has been noted and recorded in memory.

The noted image location, such as its coordinates, remains valid for reference in later images that are obtained, provided that the position of the patient remains unchanged in the imaging equipment. This enables the location to be found easily in images that are taken under different conditions such as different MRI pulse sequences, different nuclear resonance frequencies, different T1 or T2 weighting, with and without phase spoiling, etc.

The stored location advantageously is used in guided biopsy procedures, and can provide a technique for collecting a tissue sample from a tissue structure that is visible only when bound to a contrast agent, after the contrast agent has washed out and the tissue feature is no longer visible. Thus a reference marker to the position of interest is available and can be employed during subsequent imaging undertaken, for example, to image the position of a biopsy tool that is being deployed in position to effect a planned trajectory, or imaging during the process of piercing the breast tissue.

The stored location can be shown as an image marker in the display of image data, for example as the contrasting image of a sphere or box surrounding the point, which can be opaque or translucent. A reticule or projection thereof, such as cross hairs, can be superimposed on the display to point up stored locations. The image marker can be variable, for example variable in size for making size and distance measurements, movable to relocate an addressed position of interest or to define a new or additional point of interest.

In additional embodiments of the invention, computer image processing is used for projecting, rotating, storing, transforming, enhancing and displaying the data and images. Among other processes enabled according to the invention, the position of noted locations of interest can be stored per se, or a marker box, sphere or reticule can be added to the image when displayed or when a projection or slice is printed. The pixel data at and adjacent to the marked location (such as a spherical subset of the larger image) can be stored in a supplemental memory area and superimposed as imported voxel data or as a distinctly hued phantom image imposed on a later image, whereby the phantom or image of the feature obtained in an earlier (or later) image under contrast agent conditions can be displayed on a different image obtained under other conditions or optionally switched on and off to examine the associated voxels in said different image, at voxel positions corresponding to those of the contrasting tissue features in the earlier image.

Other embodiments of the invention include performing a biopsy based on the stored information. The biopsy is performed by selecting a needle capable of sampling the point of interest, installing the needle in a needle holding device, positioning the needle holding device so that the needle can sample the point of interest, and inserting the needle into the point of interest. The needle holding device is positioned by using the location data collected during the MRI scan. It is possible to locate the needle holding device at an arbitrary position relative to the target tissue and still to calculate a trajectory from the needle holding device, by placing a fiducial marker on the needle holding device and imaging the fiducial marker so as to determine the position from which the needle will be deployed. Furthermore, without re-imaging the needle holder, one can account for displacement of the needle holder by providing and appropriate displacement sensor by which the direction and extent of movement of the holder from a previously imaged location can be measured and input to the process that calculates the needle trajectory. Further, displacement of the needle holding device can be sensed using a position sensing and display unit (PDU).

In general, it is an object of the invention to cross reference one or more points in an imaged volume of a patient to one or more points apart from the patient associated with the location and trajectory of a tool. The noted points provide reference points that can be related to one another later, in particular including at least one fiducial marker that serves as a point of reference on the needle holding device.

This and other objects are met by placing at least part of a patient to be imaged, at a fixed position relative to a support or relative to a movable object, and determining relative spatial positions and orientations among two or more of the patient, the support and the object by reference to identifiable points of reference on such patient, support or object. The support can be the patient supporting table of an imaging system and the movable object can be a needle holder. Absolute or relative positions of the patient, the support and/or the object are made known relative to one another by providing images that encompass the points of reference. Accordingly, a portion of the patient is imaged to produce a volumetric data image. At least one point of interest in the data image is noted and the location of the point of interest is stored. Thereafter, by equating the point of interest as noted in the image with its position relative to other points of reference associated with the support or with the movable object, a modality can be applied to the patient at a location on or in the imaged portion that is positioned by reference to the other points of reference. It is not necessary to locate the point of interest in another volumetric data image because the point can be found by its location relative to the other points of reference. An exemplary application is noting the location of a tissue anomaly found in the breast during presence of a contrast agent, and finding the correct location of the anomaly in the physical frame of reference after the contrast agent has dissipated and the anomaly cannot be seen or seen as clearly in a subsequent image.

A number of refinements and applications are possible and are discussed herein. Additional embodiments of the invention include, for example, the use of a contrast agent to enhance the image quality and spiral or RODEO method MRI, and to employ the technique to guide tissue biopsy and/or later to confirm that the tissue collection for the biopsy was successfully accomplished at the point of interest, by similar location cross referencing or by subsequent imaging.

After a successful targeting guidance, a needle is inserted to the breast aiming for the selected (marked) lesion. A verify image is taken before the tissue is extracted. In this image, the overlap of the marker (selected lesion) on the needle indicates the relative position of the selected lesion and the inserted needle. This allows the practitioner to quickly assess the accuracy of the targeting and decide whether or not to proceed with tissue extracting.

### BRIEF DESCRIPTION OF THE DRAWINGS

The drawings show certain embodiments of the invention that are discussed herein as examples. However the invention is not limited to these examples. Reference should be made to the appended claims to determine the scope of the invention. In the drawings,

FIG. 1 shows a prior art device for holding the breast during imaging and biopsy. The device is representative of a device that can be used; however, other similar devices can be used to position the breast.

FIG. 2 shows a breast placed in the device, looking at the side of the breast.

FIG. 3 shows a conventional needle delivery device with a needle. The needle delivery device can taken on a variety of forms of which this is representative. The needle delivery device can be moved relative to the patient along all three axis and may rotate freely in multiple directions.

FIG. 4 shows a breast with a lesion in the breast holding device and the needle removing a sample of the lesion for later study. The needle is shown at the lesion, while sampling the lesion. The needle is then removed and the lesion sample is studied.

FIG. 5 shows a representative view of marking the fiducial marker and a point of interest. The view is represented to the user on a display device. Prior to marking the point of image the circle would not appear on the display device. The figure also shows a lesion, which is marked as the point of interest. The fiducial marker is marked by a rectangle.

FIG. 6 shows a flow chart of one embodiment of the invention. Not every step noted in the flow chart is necessary for every embodiment of the invention. In addition, the steps occur in this order for one embodiment of the invention. The steps representing different embodiments can be performed in a different order in conformity with this disclosure.

FIG. 7 shows the positional display unit, noting a set of Cartesian coordinates that correspond to where the needle delivery device is located. The device will note the coordinates of the needle delivery device as it is moved. The device can be located in proximity to the needle delivery device or can be located elsewhere. An onboard position sensing device can be replaced by a computer coupled to appropriate signal generation elements coupled to provide a signal that represents motion and is integrated by the computer to arrive at current position.

FIG. 8 shows a representation of the device as a whole, being used on a patient in the method disclosed. The patient is shown in the prone position with the right breast engaged in the breast holding device. A needle delivery device with a needle is shown performing a biopsy on a lesion. A computer screen represents the display device and the marked point of interest.

### DETAILED DESCRIPTION

FIG. 8 shows the main elements of an exemplary embodiment of the invention, wherein a patient 1 is placed in a prone position on a supporting table 3 or other suitable support. The patient is supported at the shoulders and torso; however a gap or opening in the support permits the breasts 5 to depend downwardly, presented for imaging. The supporting table is arranged so that the patient is held stationary relative to the table. The table can be translated into and out of the coils of a NMR/MRI imaging apparatus 33, show as a cabinet in FIG. 8. In the lumen of the coils (not shown in FIG. 8), the breasts are imaged. When the table is retracted to move the patient back to the position shown in FIG. 8, the patient is accessible for various procedures, including imaging-related activities to assist in procedures and to exploit location sensitive modalities such as the injection of a contrast agent, diagnostic procedures such as biopsy, therapeutic targeted application of nuclear or thermal radiation, or acoustic energy, etc.

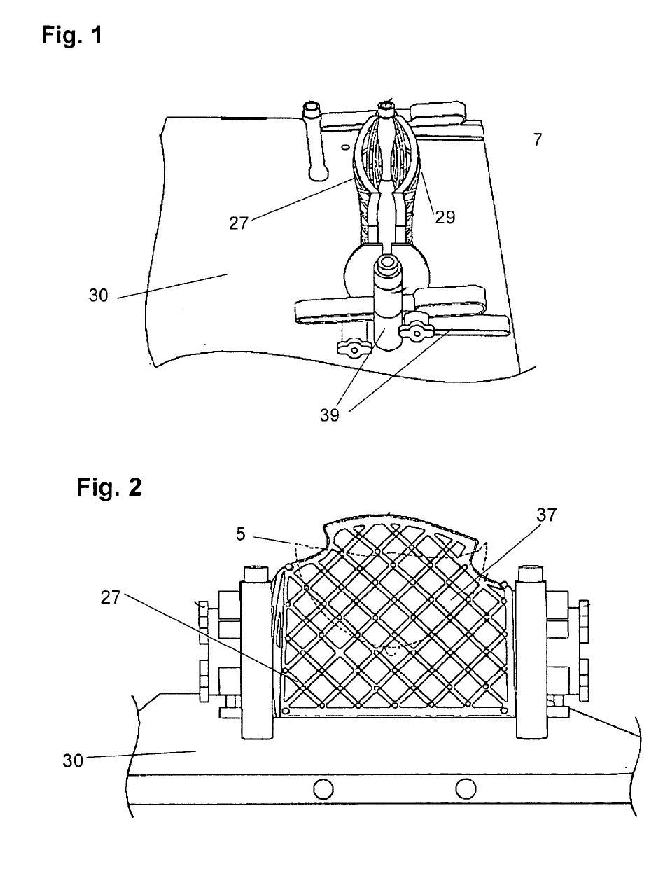

The breast 5 is immobilized relative to the table by a breast holding device 7, fixed in position on the table as shown in FIG. 1. The breast is held in a fixed position relative to the table through plural phases of the foregoing imaging, diagnostic, therapeutic and other procedures. Thus, successive imaging processes are possible wherein the breast holding device 7 and the breast 5 held therein remain in position and can be imaged before and after different procedures are undertaken. For example, the breast can be imaged to determine a target location for biopsy, and the biopsy tool positioned. The patient is imaged again, now including the biopsy tool or its fiducial markings in the collected image data. The trajectory of the biopsy tool (or other targeted mode) can be assessed, or in the case of an inserted biopsy tool, accuracy of positioning can be assessed by imaging the tool within the tissue.

At least one fiducial marker is preferably provided on the needle holder 43 as shown in FIG. 4 and when imaged defines a point of reference for the needle holder during imaging. The patient is imaged as well. So long as the relative positions of the patient and the imaging system are known (and optionally remain unchanged), it is possible to determine the location of a given point within the patient tissues, and to define a trajectory from any arbitrary position of the needle holder as indicated by a sensed fiducial marker, to intersect the location in the patient tissues.

In FIG. 8, a similar marker 9 can be provided as a point of reference on a patient support table 3. In FIG. 4, a fiducial reference point is also possible using a point on a breast holding device 7. At least one fiducial marker that has a fixed position relative to the imaged patient tissues during a time span of interest can be imaged and used to provide a reference for determining the position of a predetermined point in the tissues at a different point in time. These markers are optional if one uses an origin defined by the image system, such as the iso-center of the gradient coils, as a known location referenced to a stationary patient. In that case, only the arbitrary position of the tool or needle holder needs to be input, which can be accomplished by imaging and if necessary adjusted by position displacement measurement input. An accurate trajectory is then calculated to intersect the patient target tissue.

In a preferred arrangement, relative positions of a point in the patient tissue where a lesion has been located in an image, and a point on the needle holder at which a fiducial marker is provided likewise located in an image, provide two points whose relative positions are known. The imaging system has an inherent center at the iso-center of the gradient fields that defines a common point of reference for images that are obtained.

After determining the location of a tissue target in a volumetric image, the location in the same or a later volumetric image of one or more fiducial marks on the holder is noted and stored. This can be done using a computer processor 13 to note the associated voxel addresses and optionally a display unit 15 to the MRI image containing a depiction of the fiducial marker as a reference. A point of interest 17 can be noted using operator input, and stored by arbitrary selection or with the aid of programmed image analysis process. In FIG. 8, a point of interest 17 is marked, for example, using a computer processor 13 to note and store a voxel address. The point of interest can be chosen as a point in the tissue visually noted on a display unit 15 due to contrast with surrounding tissue in the image. Thus, the point of interest 17 might be a lesion, a tumor, a cyst or another tissue feature, the character of which may or may not be known.

One or many points of interest might be noted and stored by computer processor 13, optionally together with descriptive information regarding each point, or the regarding the patient, or regarding the imaging conditions or the like. In an advantageous application, for example, a point of interest is imaged at spaced points in time before, during and after application of a contrast agent. A tissue feature at the point of interest might not appear at all absent the contrast agent, appear with strong contrast when the contrast agent is present, and fade away within a short time due to wash-in and out of the tissues. The coordinates of the point of interest are noted, preferably when the contrast agent is present. This same point can then be found and presented in one or more other images, obtained at times before or after the application of the contrast agent, and even when the tissue feature is not seen or is less than prominent.

This location data can be retrieved and used to perform a guided biopsy, namely to obtain a tissue sample at a three dimensional point in the tissue volume, even though the target of the biopsy can no longer be seen. In FIG. 8, the guided biopsy is performed based on the coordinates determined by the imaging technique discussed above. The guided biopsy is performed using a needle holding device 21 and a needle 23. The needle holding device 21 is movable relative to the patient 1 and bears a fiducial marker 9. The needle holding device 21 can be linked to the computer processor 13 or a position display unit (PDU) 25 so that after the fiducial mark on the holder has been located, the needle holding device 21 can be re-positioned relative to the point of interest 17 and the trajectory from the repositioned location used to determine the new trajectory of the needle 23. The computer processor 13 notes the position of the needle holding device 21, the length of the needle 23, the relative location of the point of interest 17, and calculates how the needle holding device 21 as currently placed is to be oriented and the distance by which it is to be advanced. The position display unit 25 can provide numeric positioning data of where the needle holding device 21 or the collection end of the needle is located or the extent to which is has been displaced. The position display unit 25, and optionally also a displayed image, help to correctly position the biopsy tool. An image can be obtained and displayed when the biopsy tool has pierced the tissue and can be seen in projections of the MRI image, to confirm correct positioning relative to a displayed marker indicia 47 superimposed on the image projection by the computer even if the tissue feature 17 cannot be seen.

The displayed indicia can mark the tissue targeted for biopsy or otherwise can guide placement of the biopsy tool, for example to define a point of insertion, to orient the trajectory, to determine the insertion depth of the needle or the orientation of the needle holding device 21. Once the needle holding device 21 is positioned correctly and the needle 23 has been inserted up to the point of interest 17, a sample is collected and the needle is withdrawn. Subsequent imaging techniques with or without contrast agents, preferably using the marker indicia 47 and the position display unit 25, can ensure that the point of interest 17 was properly sampled.

Only one sample can be taken at a time, however, multiple points of interest can be marked and their positions stored in a single session. Standard safety precautions, procedures, and hygiene practices can be followed and are known in the art. Although in the foregoing description the points of interest are generally described as tissue anomalies, the points can also be reference points for other purposes, such as arbitrary points to assist the physician in establishing a frame of reference, points that reference nominal tissue structures such as points marking the progression of a duct, etc. Also, although the visual markings superimposed on the display are generally used to mark a point such as a single voxel in a three dimensional array, it is advantageous if the superimposed display marker appears as a larger volume centered around the point that is identified. For example, a phantom sphere or cube in a contrasting color can be shown centered on the point. The marker can be a predetermined size, such as ten voxel positions on a side or in diameter. The size of the marker can be variable according to user input. As further refinements, the superimposed marking can also bear graduation markings for purposes of measuring features shown in the image.

The breast holding device 7 of the invention can take a variety of forms, some being known in the art in connection with MRI and other imaging applications such as ultrasound or X-ray mammogram. U.S. Pat. No. 7,171,256 discloses a suitable breast holding device and is hereby incorporated by reference. The structure of the breast holding device 7 should allow access to the point of interest 17 by a biopsy needle 23, and accordingly can define an open grid of rectangular openings or a surface with spaced holes. In order to securely fix the position of the breast, an in part to widen and flatten the imaged tissue, the breast holding device 7 preferably compresses the breast 5 to an extent that is relatively snug and is maintained during the procedures. The breast holding device 7 in one embodiment has two curved paddles 27, 29 and a base plate 31. The base plate 31 is moveable relative to the patient 1 in three dimensions. The paddles 27, 29 are adjustable in position and spacing so as to fit various anatomies. Accordingly, the paddles 27, 29 preferable can be rotated and spaced at varying distances apart.

The needle delivery device 21 can be an MRI compatible needle delivery device of the sort that is known for use in connection with biopsy apparatus. The needle delivery device 21 has the additional feature that its location is defined by detection of a fiducial marker thereon during imaging and optionally can be displaced from a known location by a measured distance and direction. The needle delivery device 21 has a known geometry and range of motion, so that such geometry can be taken into account for calculating or otherwise setting the path of the needle 23 so as to intersect the point of interest 17. Further, the needle delivery device 21 can be manually operated or operated by remote control from the controller of the MRI system.

The needle delivery device 21 holds a needle 23 on a moveable fixture such the needle 23 can be aimed and advanced to the point of interest 17 by the required depth to place the tissue sampling structures at the point of interest 17. The aiming can be under computer control or by the operator reading from a computer output the required orientation and displacement values, and effecting such values by manual adjustment. The needle holding device 21 provides stability and support for the needle 23 and allows for accurate aiming and positioning of the needle 23, substantially free of human judgment and guesswork.

The needle 23 is a biopsy needle of a length and size that depend on the location and nature of the point of interest 17. The operator can select a type of needle 23 to best accomplish the biopsy. The needle 23 can be MRI compatible so as to be visible itself during imaging if the needle 23 will be used while the MRI device 33 is actively used to verify positioning at the addressed point of interest stored in memory.

The point of interest 17 preferably is selected by the user, but by appropriate image processing steps, the voxel data can be analyzed to suggest candidate points that have some attribute of contrast in images taken using one or more pulse sequences. The point of interest 17 can be at any region in the volume displayed on the MRI image and can be displayed in a projection as a region of arbitrary size and shape.

In practical application, the point of interest 17 can be a suspected lesion, a cyst, a tumor, or an unknown tissue feature that the operator wants to target, especially for biopsy. Multiple points of interest can be targeted during a single session. The points of interest can be marked with individual display indicators. The indicators 35 can have the same or different shape, size, graphical aspect, color or other attribute that an operator can recognize to identify, locate, measure, assess, segregate or organize one or a number of points of interest. The operator marks the image using software loaded onto the computer 13 by highlighting or clicking in the point of interest 17, preferably from two rotated perspectives from which reference lines normal to the image projection plane cross at the point of interest within the breast.

The imaging of the breast 5 can be performed by known MRI imaging apparatus generally depicted as device 33 in FIG. 8. In one embodiment of the invention, the breast 5 is imaged at one time as a volume (rather than successive slices) using a pulse and gradient progression encoding successive spirals. A rotating off-resonance (“RODEO”) spiral method can be used to suppress the representation of fat tissue, to enhance the contrast of water concentrations, and importantly to provide high contrast for concentrations of a paramagnetic contrast agent such as a gadolinium compound, that binds preferentially albeit briefly to lesions, tumors or other tissues that advantageously occur at the point of interest 17.

The position display 25 unit is a device that senses and can display or signal changes in the position of the needle delivery device 21. The PDU 25 can be linked to the computer 13 so that when the needle delivery device is moved, the resulting displacement data is provided by the PDU automatically. In this way, the current location of the needle delivery device is known relative to a reference point as the needle delivery device is moved, without re-imaging to determine the new device location. This information can be used to determine the trajectory and displacement of the needle delivery device needed to advance to intersect a point at which tissue is to be collected. The trajectory can accurately intersect a tissue feature that was noted by the operator at some earlier point in the imaging process, even when the tissue feature is no longer visible. The PDU 25 determines the displacement of device 21. The computer can relate the position of the device 21 and the targeted tissue feature to one another because a reference origin and orientation are associated with the imaging apparatus or are associated with a fiducial marker on the device 21 that appears in at least one image. Optionally, additional fiducial markers can be used as alternative location references for the patient support table, the breast restraint, etc., as desired. Preferably, however, the imaging system provides a location reference that applies to all images it collects, and the fiducial marker on the device 21 enables aiming of the biopsy needle or other tool.

The method is advantageously practiced using a computer processor such as a PC coupled to an image memory and arranged to operate or trigger or monitor signals on the bias and gradient coils, and the MRI receiver. The processor or an associated image acquisition controller can produce various pulse sequences associated with different image weighting and similar techniques. The processor can manage the associated math including Fast Fourier Transforms to obtain voxel values, data value normalization and gain control, application of thresholds, mathematical image data enhancement, projection of the 3D voxel data to a 2D display, rotation of the projection and so forth. The computer system 13 is coupled to a display 15, and comprises a processor, a memory and a user interface (not shown). The computer 13 can be loaded with software that controls the apparatus for collecting MRI data, or can be coupled to data representing an already-acquired magnetic resonance image that allows the user to mark the fiducial marker 9 and the point 17 or points of interest and that calculates the relative position of the point of interest 19 relative to the fiducial marker 9 as described.

The position or location data is displayed, stored, and can be printed in hardcopy form. In addition, in an embodiment of the invention, the location data 19 is combined with the location of the needle delivery device, the geometry of the needle, and the geometry of the needle delivery device. Once the combined data is processed, the computer 13 determines where the needle delivery device 21 must be aimed and advanced based on the target location. The operator sets the needle delivery device 21 to the prescribed trajectory and performs the biopsy.

When employed during the perfusion of imaged tissue with a contrast agent, the location of the point of interest 17 can be found and recorded at a time of relatively higher contrast before the contrast agent washes out and its image effects fade away. However once the location of the point of interest 18 is recorded (stored in memory either directly or by data from which the point an be derived), it is no longer necessary to view the tissue image in order to make use of the marked location of the point of interest. Thus the point of interest can be resolved in later images after the tissue feature is no longer seen for biopsy, or in pre-contrast images for comparison or manipulation of contrast or post-contrast image data, e.g., to guide the biopsy, ensure a correct location for tissue collection and examine the results afterward.

In FIG. 1 a device for holding the breast 7 during imaging and biopsy is shown. The device 7 is representative of known type of holder, and other similar devices can be used to restrain and position the breast. The opposed paddles 27, 29 are connected to a base plate 31. The paddles 27, 29 have openings 37 that provide access to the breast skin and tissue. Articulations 39 for adjustments are also shown.

FIG. 2 schematically shows a breast 5 engaged in the holder device 7. The view is a side view of the breast 5 and only shows a paddle 27 and the openings 37. The openings 37 are large enough for the needle 23 to pass through. The paddles 27, 29 preferably are substantially transparent to MRI, but also can carry one or more fiducial markers (not shown) at a predetermined location thereon.

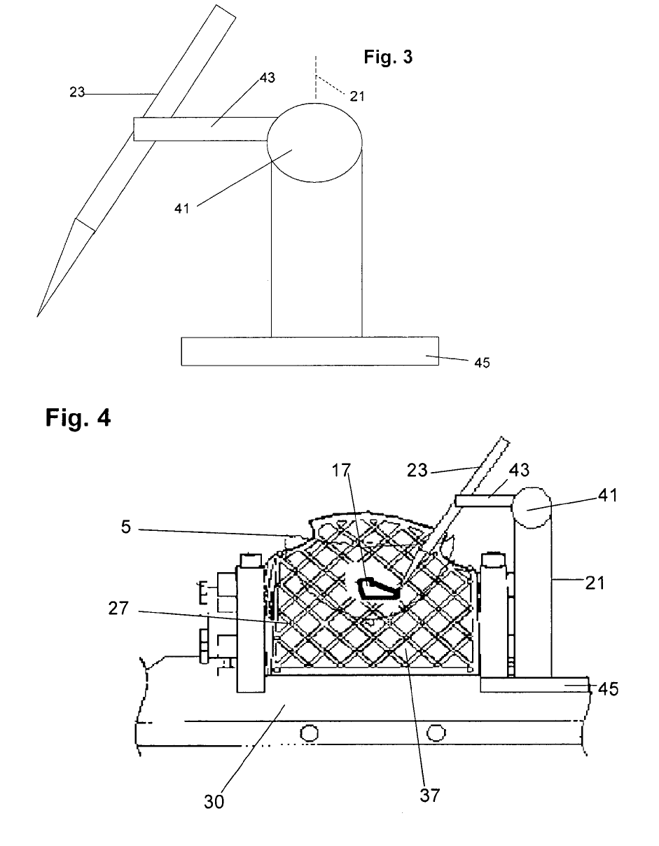

FIG. 3 schematically demonstrates the elements of a needle delivery device 21 carrying a needle 23. The needle delivery device 21 has various degrees of freedom by which device 21 can be brought into proximity to one of the breast paddles, and needle 23 can be oriented on a line between an initial point of entry through an opening in the breast paddle, and the internal point at which the tissue is to be collected in a biopsy procedure. Preferably, the needle delivery device 21 can be moved relative to the patient 1 along all three axis and may rotate on its axis or on a universal joint with an attachment base 45 or otherwise. When in position, the needle 23 and holder 21 are fixed relative to the breast paddles and relative to the patient support table. For purposes of illustration in FIG. 3, the needle 23 can rotate at a pivot 41 so the needle 23 can be placed to avoid interfering with the paddles 27, 29 when penetrating the breast.

If the holder 21 and/or needle 23 are allowed to translate or rotate, it is appropriate input is provided to the computer 13 to reflect new location data. This can be by manual data entry but preferably is by automatic data capture using displacement measuring devices. After repositioning, the trajectory and depth to the target must be recalculated and confirmed. The needle delivery device 21 in an embodiment of the invention has a needle holder 43, where the needle 23 is placed and has an attachment 45, which supports the needle delivery device.

FIG. 4 is partly cut away and shows a breast 5 in the breast holding device 7 with a point of interest 17 and the needle 23 removing a sample at the point of interest 17 for later study. The needle 23 is shown at the point of interest 17, while sampling the point of interest 17. The needle 23 is then removed and the point of interest sample is studied. The needle 23 is held in the needle holding device 21. This arrangement is exemplary with respect to various aspects such as the precise position of holder 21 and the orientation of needle 23.

FIG. 5 shows a representative view wherein the imaged tissue including the point of interest 17 and a fiducial marker 9 appear in the same view. The view is a 2D projection or slice, presented to the user on a display device 15. Prior to recording the location of a point of interest 17 no marker such as crosshair oval 47 appears. After a location is noted and stored, such a marker can be superimposed on the display device 15, as can a similar marker 45, the preferably is visually different from crosshair oval 47. The point 17 as marked could represent an arbitrary point or tissue structure but preferably is used to mark a tumor, lesion, cyst or the like.

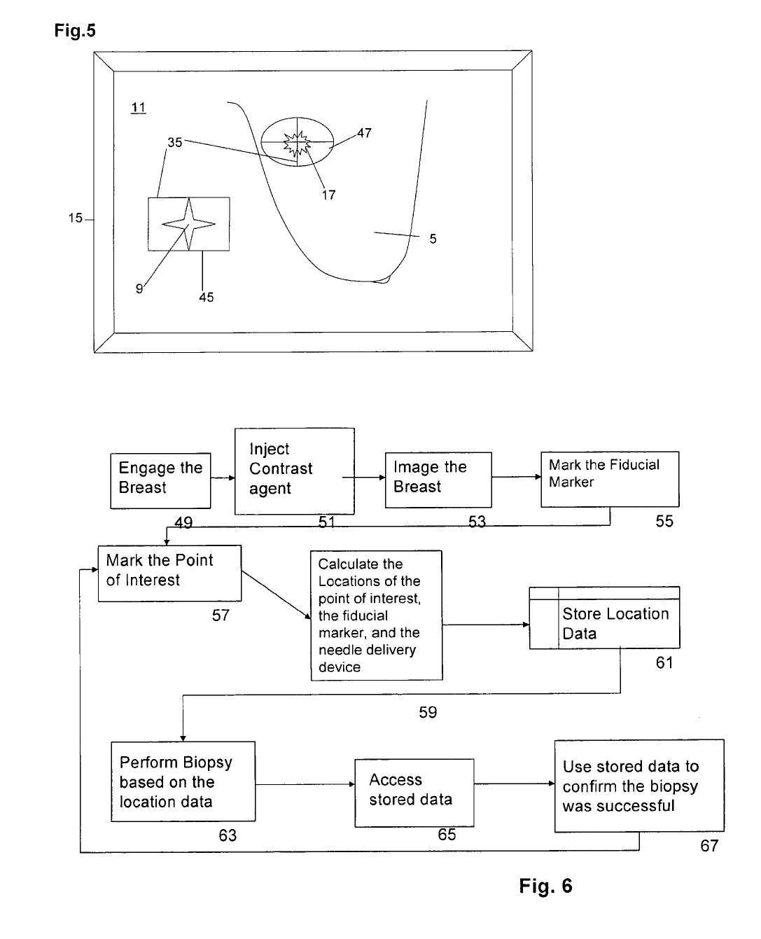

FIG. 6 is a flow chart representing a method that can be followed according to one embodiment of the invention. Not every step noted in the flow chart is necessary for every embodiment of the invention. Likewise, additional steps and the reordering of the steps maybe possible in a given situation, while in conformity with this disclosure. The breast 5 is inserted at 49 to the breast holding device 7 and a contrast agent is in injected at 51. The breast 5 is imaged at 53. The location of the fiducial marker 9 is marked at 55 and the location of the point of interest 17 is marked at 57. The location of the fiducial marker 9, the point of interest 17, and the needle delivery device 21 are related to one another by calculations at 59. The location data is stored at 61 and the biopsy is performed at 63. The data is retrieved at 65 and the breast 5 is imaged again at 69 to assess the tissue collection and whether the biopsy was successful. Only noting the point of interest 17 is necessary when the contrast agent is actively affecting the image. The remaining steps can be done after the contrast agent has faded. The process is repeated from the step of marking the point of interest 57 for studies involving multiple points of interest.

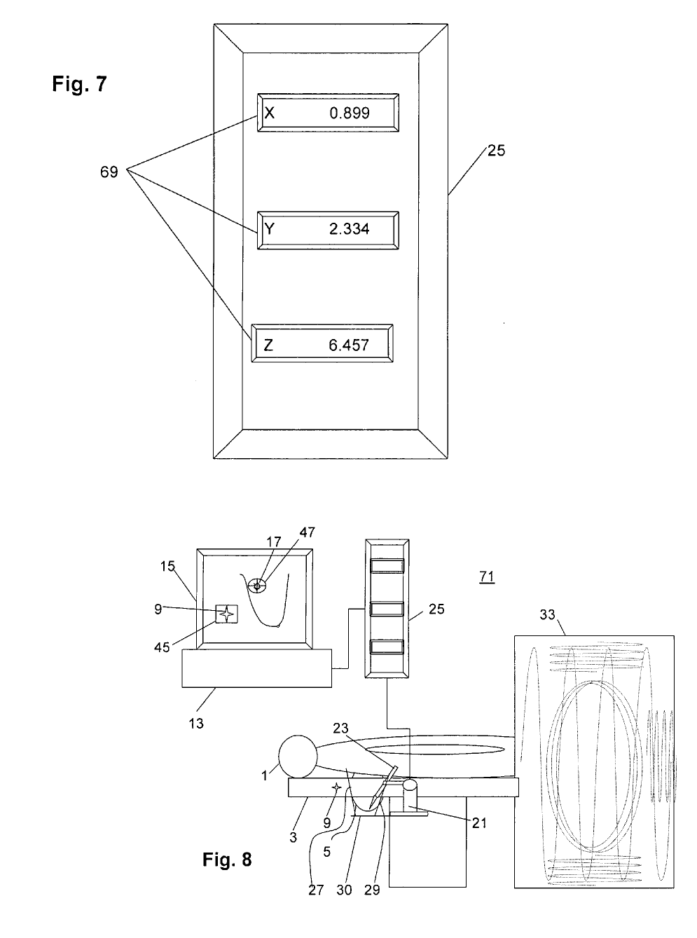

FIG. 7 shows one possible arrangement for a positional display unit 25, in this case comprising a readout 25 of coordinates that can be Cartesian coordinates, for example, corresponding to where the needle delivery device 21 is located along mutually orthogonal X, Y and Z axes. With appropriate sensing inputs, the PDU 25 can reflect changes when the needle delivery device 21 as it is moved. The PDU 25 can be located in proximity to the needle delivery device 21 or can be located elsewhere. The PDU 25 can be replaced by a computer. The connections are not shown.

FIG. 8 has been discussed to some extent above and shows a representation of the device 71 as a whole, being used on a patient 1 using the method disclosed. The patient 1 is shown in the prone position with the right breast 5 engaged in the breast holding device 7. A needle delivery device 21 with a needle 23 is shown performing a biopsy on a point of interest 17. A computer screen represents the display device 15 and the marked point of interest 17. The computer 13 is also shown, as is the PDU 25. The patient 1 has been removed from the MRI device 33 but remains on the examination table 3.

An aspect of the disclosed technique is to enable a point within the volume of the patient to be located by providing positioning information. This can include causing a fiducial marker is caused to appear in two images such that the marker provides a common reference point that individually or with other aspects enables relative positions to be determined in three dimensions in both images. Alternatively the imaging system can be deemed to define an origin and an orientation that remains fixed in a physical frame of reference in which the imaging system is located. The positioning reference point or origin, and also the orientation of the physical frame of reference in that case can be the gradient iso-center of the magnetic field coils. By establishing a positioning reference that is applicable to two or more images, a feature that may be visible temporarily at an arbitrary location in an imaged volume of tissue, can be found in another image, even under circumstances where the attribute that rendered the feature visible is not present. Likewise, having established a dependable way to determine positions, a biopsy collection or apparatus can be deployed at a desired location and/or displaced in a metered way and then deployed.

The foregoing disclosure and associated drawings represent embodiments that are exemplary and may be preferred in some situations but are not all inclusive. Reference should be made to the appended claims to assess the scope of the invention in which exclusive rights are claimed.

What is claimed is:

1. A method for volumetric imaging of a patient, comprising:
placing at least part of a patient so as to fix in position a portion of the patient to be imaged;
imaging the portion of the patient to produce a volumetric data image;
noting at least one point of interest in the data image, and storing a location of the point of interest;
determining a location of a modality at least partly from a subsequent imaging operation wherein the modality is detected, the subsequent imaging operation having a known position relative said imaging of the portion of the patient; and, determining a relationship of the modality versus the location of the point of interest, and applying the modality to the patient at the point of interest.

2. The method of claim 1, wherein the modality is sensitive to location and comprises at least one of tissue collection by biopsy, application of a targeted injection, and application of targeted energy to an affected point within said portion of the patient.

3. The method of claim 2, wherein the modality comprises a tissue collection device located at a position detected by imaging said device, and further comprising determining a trajectory to the point of interest.

4. The method of claim 2, further comprising applying a contrast agent to enhance contrast of a tissue feature when noting said point of interest in the data image, and wherein said modality is applied to the patient at the point on interest, using the location reference established from the fixed point, at a time after said contrast has substantially dissipated.

5. The method of claim 2, further comprising imaging the portion of the patient to produce at least one other volumetric data image before or after said volumetric image from which the point of interest was noted, and applying to the other volumetric data image a marking indicia identifying the location of the point of interest.

6. The method of claim 5, wherein at least one said point of interest in the data image represents a tissue anomaly and wherein the marking indicia identifying the location of the point of interest is applied when the tissue anomaly is not otherwise visible.

7. The method of claim 3 further comprising displacing the modality after determining said location of the modality by said subsequent imaging operation, determining a displacement of the modality from the location determined by the subsequent imaging, and determining said relationship of the modality versus the location of the point of interest according to said displacement, before applying the modality to the patient at the point of interest.

8. The method of claim 3, further comprising noting in the data image a location of a fiducial marker, wherein the location of the point of interest is stored so that the location of the point of interest can be found relative to the location of the fiducial marker.

9. A method for one of diagnosing and treating mammalian breasts, comprising:
placing a breast at a predetermined position;
collecting a volumetric data image of the breast in which tissue structures are represented by voxel data values in a three dimensional matrix;
displaying a two dimensional image by producing a display presenting at least one of a planar slice through the volumetric data image and a plurality of projections of the volumetric data image;
identifying a point of interest within the breast by viewing the display and determining at least one locating point in the three dimensional matrix from which the point of interest can be derived;
storing an address of the locating point;
collecting at least one additional volumetric data image of the breast in which tissue structures are represented by voxel data values in a three dimensional matrix;
correlating the point of interest from said volumetric data as identified by viewing the display, with a corresponding location in the additional volumetric data image; and,
conducting at least one operation using the corresponding location of the additional volumetric data image as an applicable point of interest.

10. The method of claim 9, wherein at least one said operation comprises displaying a two dimensional image by producing a display presenting at least one of a planar slice through the volumetric data image and a plurality of projections of the additional volumetric data image, and superimposing on the display an indicia identifying the applicable point of interest.

11. The method of claim 10, wherein the operation comprises deploying a modality operable on a target location, and further comprising providing a fiducial marker on said modality, arbitrarily placing the modality relative to the target location, detecting the fiducial marker, and calculating from a detected location of the fiducial marker at least one of a trajectory and distance to the target location.

12. The method of claim 11, wherein the operation comprises a breast biopsy and the operation comprises directing a biopsy tool from a holder to the target location.

13. The method of claim 11, wherein the modality is directed to a location within a human breast and comprises at least one of tissue collection by biopsy, application of a targeted injection, and application of targeted energy to an affected point within said breast.

14. The method of claim 13, wherein the modality comprises tissue collection at a point associated with the point of interest, and further comprising guiding the point of tissue collection relative to an arbitrary location of a holder supporting the modality.

15. The method of claim 13, further comprising applying a contrast agent to enhance contrast of a tissue feature when noting said point of interest in the data image, and wherein said modality is applied to the patient at the point, by locating the point after said contrast has substantially dissipated.

16. The method of claim 13, further comprising imaging the portion of the patient to produce at least one other volumetric data image before or after said volumetric image from which the point of interest was noted, and applying to the other volumetric data image a marking indicia identifying the location of the point of interest.

17. The method of claim 16, wherein at least one said point of interest in the data image represents a tissue anomaly and wherein the marking indicia identifying the location of the point of interest is applied when the tissue anomaly is not otherwise visible.
